(19) United States
(12) Patent Application Publication
(10) Pub. No.: US 2010/0236135 A1
(43) Pub. Date: Sep. 23, 2010

(54) SYSTEM AND METHOD FOR GROWING ALGAE ON HUMAN INFRASTRUCTURE

(76) Inventor: Oren L. Kleinberger, Talking Rock, GA (US)

Correspondence Address: GARDNER GROFF GREENWALD & VILLANUEVA. PC, 2018 POWERS FERRY ROAD, SUITE 800, ATLANTA, GA, 30339, US

(21) Appl. No.: 12/740,710

(22) PCT Filed: Oct. 31, 2008
(86) PCT No.: PCT/US08/81962

§ 371 Date: Apr. 30, 2010

Related U.S. Application Data
(60) Provisional application No. 60/984,124, filed on Oct. 31, 2007.

(51) Int. Cl. C12M 1/00; C10L 1/14; B01D 53/34

(52) U.S. Cl. 44/307; 435/266; 435/292.1

(57) ABSTRACT

A tank infrastructure that is installed on space that is covered with human development, and used to grow blue green algae or other algae. This includes application over roads, tracks, buildings, parking lots, and any other space that is viewed from space as being any color other than green due to human development destroying photosynthetic life. It can also be utilized in desert landscapes and other areas such as in or over oceans. The algae produced will act as a large carbon sink to aid in curbing global warming, and will be used in the production of alternative fuels as well as for human or animal consumption and in industry.

### CROSS-REFERENCE TO RELATED APPLICATION

This application claims the benefit of U.S. Provisional Patent Application Ser. No. 60/984,124, filed Oct. 31, 2007, which is hereby incorporated herein by reference in its entirety for all purposes.

### TECHNICAL FIELD

The present invention relates primarily to systems and methods for producing fuel or energy, for reducing carbon dioxide greenhouse gases in the atmosphere, for producing oxygen, for heating and cooling living spaces, for alleviating global warming, for desalinating seawater, for producing food, and/or for achieving any combination thereof. In an example application, the invention relates more particularly to a system and method that utilizes any space or surface area that can been seen from satellite images of the planet Earth, or which are otherwise exposed to sunlight or other photosynthesis-generating light, which are terrestrial or otherwise, and which are not currently green due to photosynthesis, for the production of biomass using preferably algae or blue-green algae; and more specifically wherein the biomass that is created is both a carbon dioxide sink and has potential uses for alternative fuel sources, human consumption, and/or other applications.

### BACKGROUND AND SUMMARY OF THE INVENTION

Growing algae such as blue-green algae can lead to much greater yield-per-acre for plant oil production than any crop that is currently grown. There are a myriad of potential uses for the biomass that is created when algae are grown and then dried and processed. Typically, algae are cultivated in large open aquatic pools or ponds, which take up space that could be otherwise utilized. When the planet Earth is analyzed from space, any highly industrialized regions are primarily grey and black in color. This grey color is caused by human development, primarily buildings, roads, parking lots, and other travel-ways. With a different model in place, and according to the present invention, every area that is currently not green due to photosynthetic life can be made green by installing open or closed tanks of blue-green algae above or on top of them. This potentially would create a cooling effect on the planet because a considerable portion of the areas that were absorbing direct radiation into asphalt or concrete could be covered with surface area that would reflect and/or absorb and efficiently utilize the sun's energy for productive purposes. This model would allow humans to utilize space that is currently wasted for the production of dried algae.

Algae is currently used in many markets and has a potential to fulfill many other human needs as production increases. It can be used for the production of biofuels and plastics as well as in food and in skin-care products. Algae production can also serve as a substantial carbon sink, which can aid the effort to lower carbon dioxide concentrations in the atmosphere, which is directly linked to global warming.

Covering roads, parking lots, buildings, and other travel-ways with vessels acting as water tanks prolific with algae would dramatically increase the life-span of the materials utilized in the construction of these objects. Weathering and solar-deterioration are the primary causes of the breakdown of many materials used to construct buildings or used in paving. If algae-growing tank infrastructure according to the present invention covered every major highway in the world, our highways would likely have a much greater life-span and require dramatically less maintenance.

Additionally, the gasses emitted by the algae can be harvested and utilized. For example, these gasses can be processed into hydrogen fuel sources and other fuel sources.

Photosynthesis is nature's solar panel. Currently, no human-made electric solar panel can compare to the efficiency of nature's photosynthesis. The Sun is the ultimate source of nearly all of the energy utilized today, including petroleum, coal, natural gas, and biofuels. It is important for humans to begin harvesting the Sun's energy using the most efficient and effective methods. Thus it can be seen that needs exist for improved systems and methods for growing algae and/or other aquatic plant life on human infrastructure. It is to the provision of improved systems and methods meeting this and other needs that the present invention is primarily directed.

The present invention provides systems and methods for growing plant life of various types, preferably algae such as blue-green algae, on top of, over, on, or otherwise supported by, structures or areas that are typically not actively seasonally green due to photosynthesis. The system can be designed in many ways to achieve the desired result, and it may be an open system or a closed system depending on the water supply in the region. This invention encompasses the concept of growing different types of algae in tanks or other containers or infrastructure mounted above or on top of human-made objects and structures. The algae is collected by one or more separators or harvestors, such as a screen, filter, rake, strainer, or other mechanical apparatus, chemical separation process, or other means.

In the closed system model, the container preferably includes a ridged transparent top, and an optionally transparent bottom and side. The tanks are preferably installed at an angle to encourage a natural circulation effect and to allow for self-cleaning surfaces using air bubbles. There is optionally a bubbler system at the lowest end of the tanks and at other points that bubble air within the container, and these air bubbles follow the ridges in the transparent top to the highest point in the tank cleaning the surface along the way. This serves to both aerate and circulate the algae as well as to keep the surface free of algal buildup so the sunlight can adequately penetrate the tanks with minimal maintenance. The tanks may be cleaned chemically, mechanically, with gasses such as ozone, or otherwise, and may also be designed to flip with an optional ridged bottom to allow the air bubbles to clean the top and bottom surfaces. If the system is a closed system, it is advantageous to keep the top surface clean in order to most efficiently utilize the sun's energy. These tanks may be designed as thin as a few inches or as thick as many feet depending on the application and placement.

The temperature of the water in the tanks can be maintained at desired or optimal levels using a variety of different methods. An effective and simple method is to control the water temperature by controlling the temperature of the air that is being bubbled into the tanks. This air can be cooled naturally by pushing it first into the earth where it can absorb the ground temperature below the surface of the ground, and it can be heated naturally utilizing solar heaters or geothermal heaters. There are many methods that can be used to heat or cool the water in which the algae live that will be designed specifically for each individual climate in which the tanks are installed. It is preferred that heating and cooling methods utilized be energy efficient utilizing local resources. The optimal temperature to be maintained will depend on the specific types of algae utilized.

Air can optionally be captured off the highest point in the tank of a closed system model. This air is found to be rich in many valuable elements and compounds and can be processed to produce hydrogen, oxygen, and other valuable resources.

Open system tanks may also be utilized, especially in areas of natural water abundance. There is also a possibility of utilizing salt water systems with salt water algae in either closed or open loop system. Each open system is uniquely designed based on the environmental constraints of the areas in which they are installed. They will preferably be fine-tuned to obtain optimal performance for each individual climate.

Other subsystems may optionally be included within the system and method of the present invention to enhance the overall function of the invention. In areas of salt water abundance, closed system tanks may be used in combination with a condensing unit to capture fresh water as it evaporates from within the tanks. This would be very useful in cities near the ocean that don't have natural fresh water abundance. An additional subsystem could also capture the air coming from the tanks to be used as high quality air for humans rich in Oxygen or for other purposes. This higher quality air can be piped into buildings or houses.

Genetic research will take place to ensure a large genetic pool of algae to work with. Different algae have different properties. Temperature range, pH range, salinity range, and other ranges have an individual alga species which will produce a maximum production yield of lipid biomass within those ranges. Additionally, it is advantageous to integrate genetic diversity in order to avoid potential population collapses due to disease and other environmental factors.

The algae are preferably piped and processed in one of two ways. One method is to pipe the algae-rich water into a routine maintenance truck that strains the algae and restores the proper nutrients and other inputs to facilitate the next batch. The other method uses pipes to pipe the oil to a facility or machine that strains and processes the algae. This may be piped a distance to a straining and pressing facility, or it may have a processor on site directly next to the tank infrastructure.

In one aspect, the invention is a method of absorbing solar energy and carbon dioxide, the method including mounting a container over a structure; exposing the contents of the container to sunlight; delivering air into the container; and growing algae within the container.

In another aspect, the invention is a system for growing algae over human infrastructure, the system including a tank comprising a sunlight-transmissive top portion; a quantity of water disposed within the tank; and means for delivering air into the tank.

In another aspect, the invention is a building system including a container of algae-containing water, the container having at least one light-transmissive surface for generating algae growth within the water. The system further includes a water collection subsystem for collecting water vapor formed from evaporation of the water, an air collection subsystem for collecting oxygen-rich air generated in the container, a processing subsystem for producing fuel from the algae, and a generator for producing power from the fuel.

These and other aspects, features and advantages of the invention will be understood with reference to the drawing figures and detailed description herein, and will be realized by means of the various elements and combinations particularly pointed out in the appended claims. It is to be understood that both the foregoing general description and the following brief description of the drawings and detailed description of the invention are exemplary and explanatory of preferred embodiments of the invention, and are not restrictive of the invention, as claimed.

### BRIEF DESCRIPTION OF THE DRAWINGS

FIG. 1 shows an open-loop system installed on a rooftop according to an example form of the invention.

FIG. 2 displays two different styles of ridged surfaces according to example forms of the invention.

FIG. 3 shows a ridged top tank according to another example form of the invention.

FIG. 4 shows a two ridge top tanks installed above a highway, according to another example form of the invention.

FIG. 5 is a tank with a ridged top and a ridged bottom and bubbling bars, according to an example form of the invention.

FIG. 6 and FIG. 7 show different variations of pipe design according to example forms of the invention.

FIG. 8 is a tank that is installed as a buffer alongside of an interstate highway, according to another example form of the invention.

FIG. 9 shows the narrow tank design acting as a wall and rooftop, according to another example form of the invention.

FIG. 10 shows a tank system installed over a body of water.

FIG. 11 shows a building with tanks filled with algae mounted to the walls and on the roof, with additional subsystems added according to an example form of the invention.

### DETAILED DESCRIPTION OF EXAMPLE EMBODIMENTS

The present invention may be understood more readily by reference to the following detailed description of the invention taken in connection with the accompanying drawing figures, which form a part of this disclosure. It is to be understood that this invention is not limited to the specific devices, methods, conditions or parameters described and/or shown herein, and that the terminology used herein is for the purpose of describing particular embodiments by way of example only and is not intended to be limiting of the claimed invention. Also, as used in the specification including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment.

The present invention provides a tank system to grow algae of different types in different climates. There are two primary embodiments of the invention, the open-loop and the closed-loop systems. Both the open- and closed-loop systems incorporate an intensive bubbling system, temperature control system, water level control system, algae removal system, and cleaning maintenance system. The open-loop system potentially has a cover to eliminate debris from entering the tank or a greenhouse type structure or roof, but these artificial ponds can also be left completely open to the atmosphere. The tank of the open-loop system may be constructed of any material(s) that is/are durable over time. The closed-loop system tank has been designed to be installed preferably at an angle with at least a clear surface made of either glass or a UV-resistant synthetic material to allow in the sunlight that is preferably ridged on at least one surface.

Both systems use tanks that can vary greatly in size and shape depending on the application. The tank is preferably between one inch and 10 feet in depth. Tanks are connected and divided in a manner that would isolate potential failures or leaks in reasonable sizes. At the bottom of the tank, there are preferably pipes with nozzles, air stones, or other bubbling devices installed to percolate the tank with preferably local atmosphere. Additional bubbling devices may be placed strategically throughout the tank in other locations to encourage circulation and algae growth. The tank temperature is preferably controlled by the temperature of the air that is inputted into the system. The air that is being bubbled into the tank serves at least three purposes: create a carbon dioxide rich environment for the algae and carry out the oxygen and other gases that may have other potential industrial uses if captured and refined; help control the temperature of the water; and keep the surface of the tank in the open system clean so that the sun rays can enter the tank. The side of the tank that is exposed to the sun is preferably ridged on the inside so that the air bubbles can follow the ridges and keep the tank clean on this surface.

Another embodiment of the invention utilizes clear pipes or conduits that are either made of glass or of a high quality UV-resistant synthetic material which act as tanks. The pipes are preferably installed at an angle and can be placed in a myriad of different arrangements. These pipes have bubbling devices installed for the same reasons explained in the first embodiments. The pipes may also be designed to rotate individually or as a group in order to encourage circulation and keep all the surfaces clean. The pipes may also be connected together in different applications and configurations or maintained independently.

Another example embodiment of the invention uses preferably smooth flat inner and outer surfaces of the tank with tanks that are tall and thin. In this embodiment, the tank serves more as a wall or buffer than a cover or roof. The tanks will typically be installed vertically, but may be at a slight angle or one surface may be at an angle. The surfaces would preferably either be cleaned chemically, mechanically, and/or with a sucker type living organism. Examples of applications for this embodiment are walls of buildings, noise or land buffers, and privacy fences.

Another embodiment of the invention utilizes a structure similar to those found in thin-walled plastic greenhouses. These polycarbonate walls and roofs are thin and are commonly made of materials such as Lexan or Verolite, which are UV resistant and allow a more complete solar spectrum to pass through. In this embodiment, the walls may be as thin as ⅛″ or as wide as two feet in depth and are preferably produced to a size that is manageable to transport. These thin sheets are filled with water and bubbles in order to grow algae that are preferably made of a high-strength glass or a high-strength synthetic material. They are cleaned regularly either mechanically or chemically. These sheets may be stacked in order to create more depth and could act as a wall, roof, fence, decoration, or stand-alone structure. These sheets may also be used in the construction of any of the other four embodiments.

Any of the embodiments may be designed with either batch-style or continuous flow-through designs. If the algae are grown in batches, the algae are extracted from the tanks at regular intervals that correlate with the maximum production yields and lifecycles of the algae. The flow-through design starts at one point with a low concentration of algae and then moves through a system of individually divided tanks steadily increasing in abundance until the algae are at a high concentration at the end of they system where they are then continually processed or harvested. In order to create flow-through design, strategic walls and air holes are placed within a tank, or different tanks or pipes are connected together.

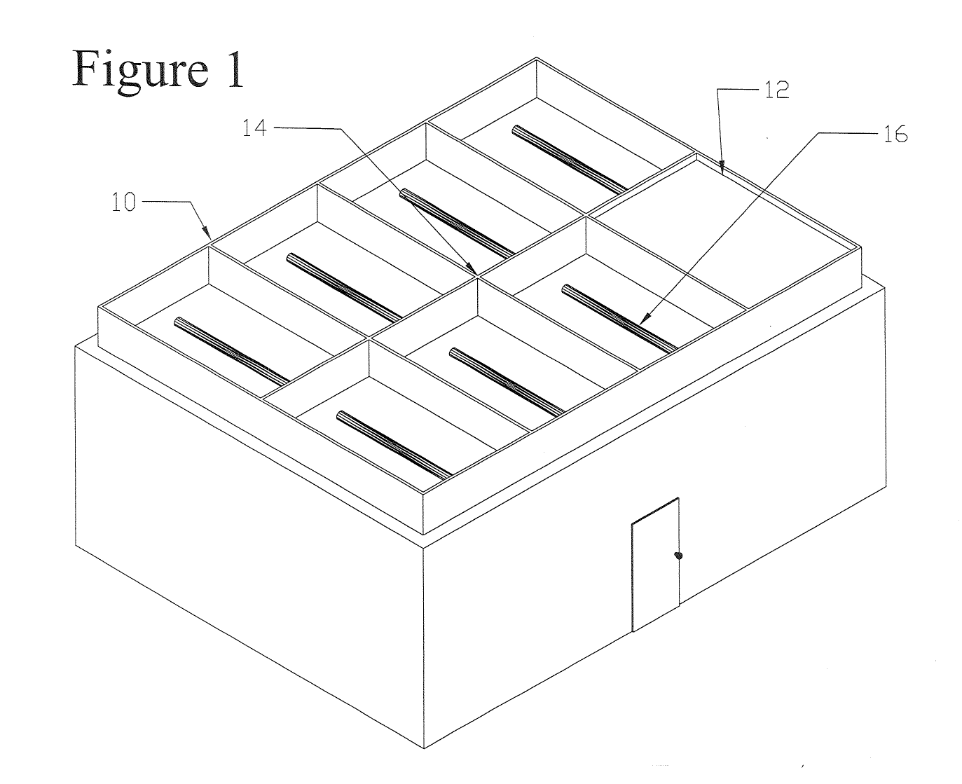

FIG. 1 is exemplary of an open system according to the present invention, where the tank 10 is filled with water that has algae living within it and the surface of the water 12 is open to the atmosphere. The tank is optionally divided with walls 14 in order to prevent the entire tank from draining if a leak should form. At the bottom of the tank, there are bubblers 16 that bubble atmospheric air and potentially industrial fumes rich in CO₂ to aid in algal growth while cleaning the air. In this example, the tank is mounted on the roof of a flat-roofed building 18.

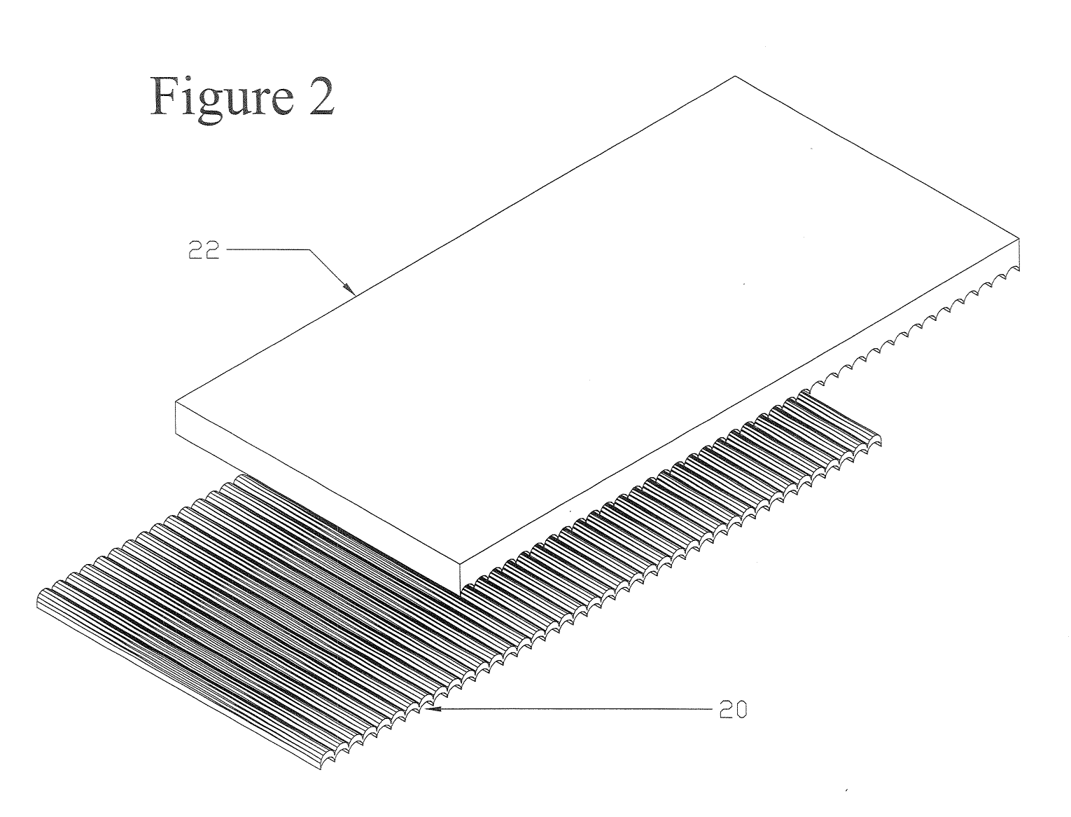

FIG. 2 shows two different styles of the ridged transparent surface that are suitable for use in closed system embodiments of the invention, which may be installed at an angle or with a wall at an angle, relative to the horizontal. This ridged design creates a self-cleaning surface when used in combination with bubbles. Different size bubbles follow the grooves and keep the surface clear of build-up that would inhibit light penetration. Element 20 is a representation of the surface that is ridged on both sides, and Element 22 is a representation of the surface that is smooth on the outside and ridged on the inside.

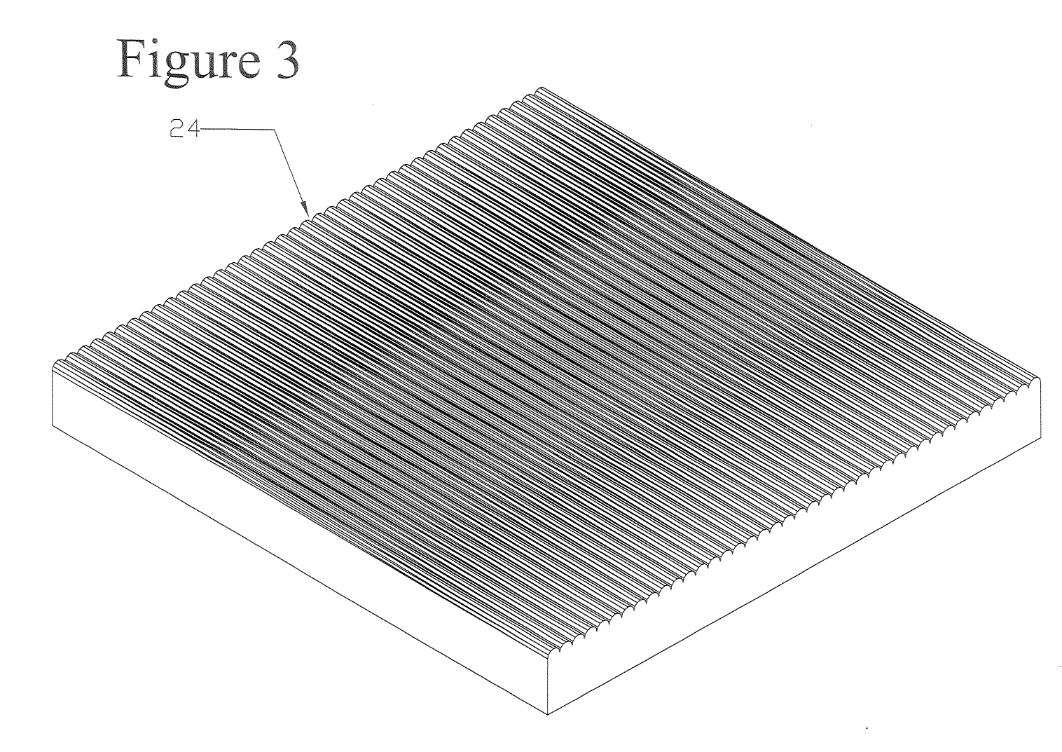

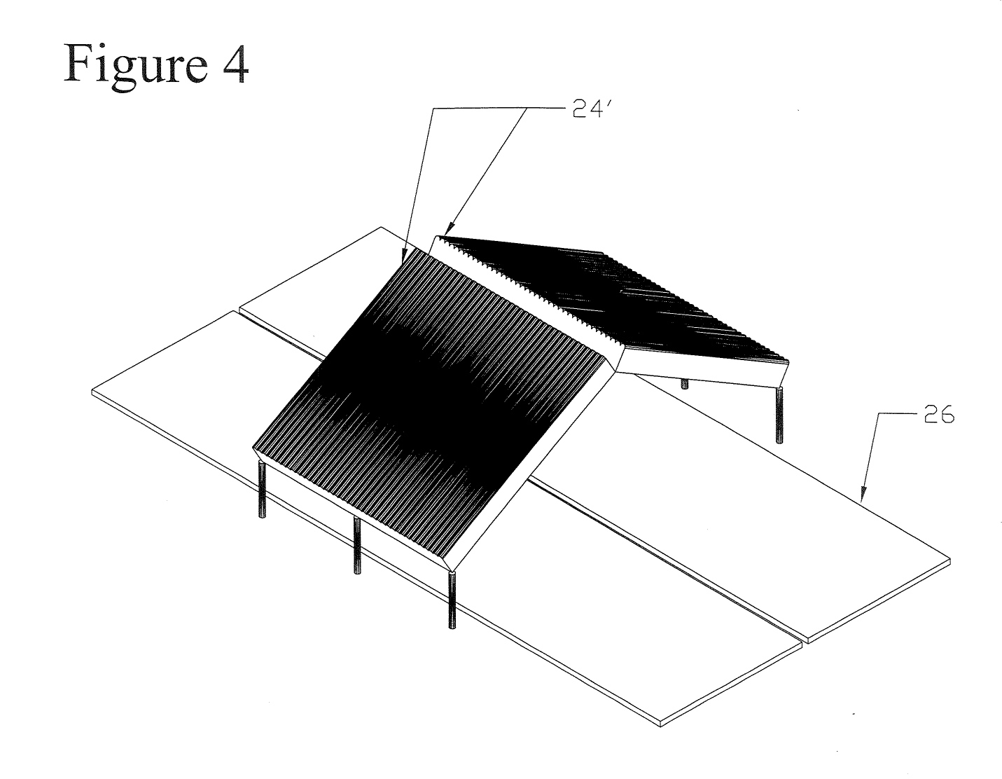

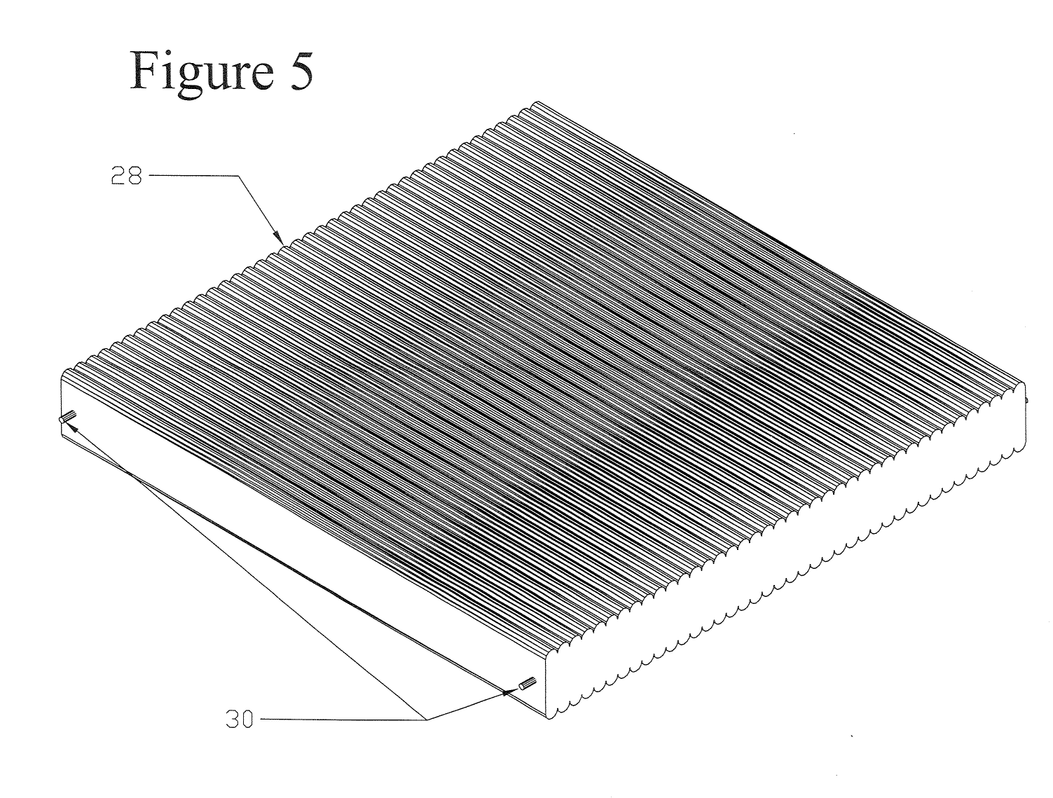

Referring to FIGS. 3, 4 and 5, alternate forms of closed-loop systems with ridged surfaces are represented. FIG. 3 shows a tank 24 where one side is ridged and transparent and the other sides are optionally transparent. In FIG. 4, two of these tanks 24′ are placed over a highway 26. FIG. 5 shows a tank 28 with a ridged top and bottom. This tank 28 can be mounted in a way that would rotate it at a time interval to keep both the top and bottom surface clean using bubbles. In this figure, bubbling bars 30 have been installed, one of many methods for creating bubbles in the tanks.

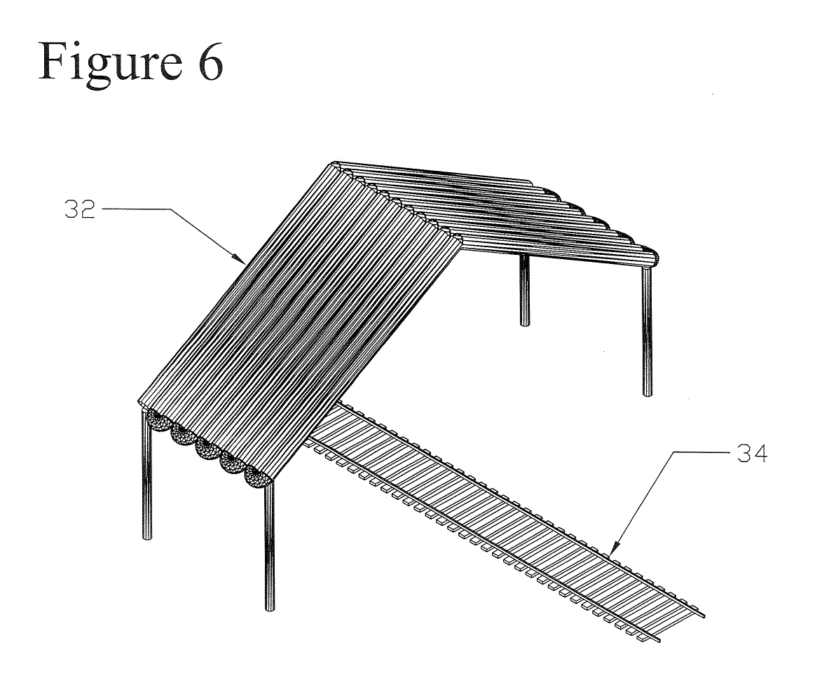

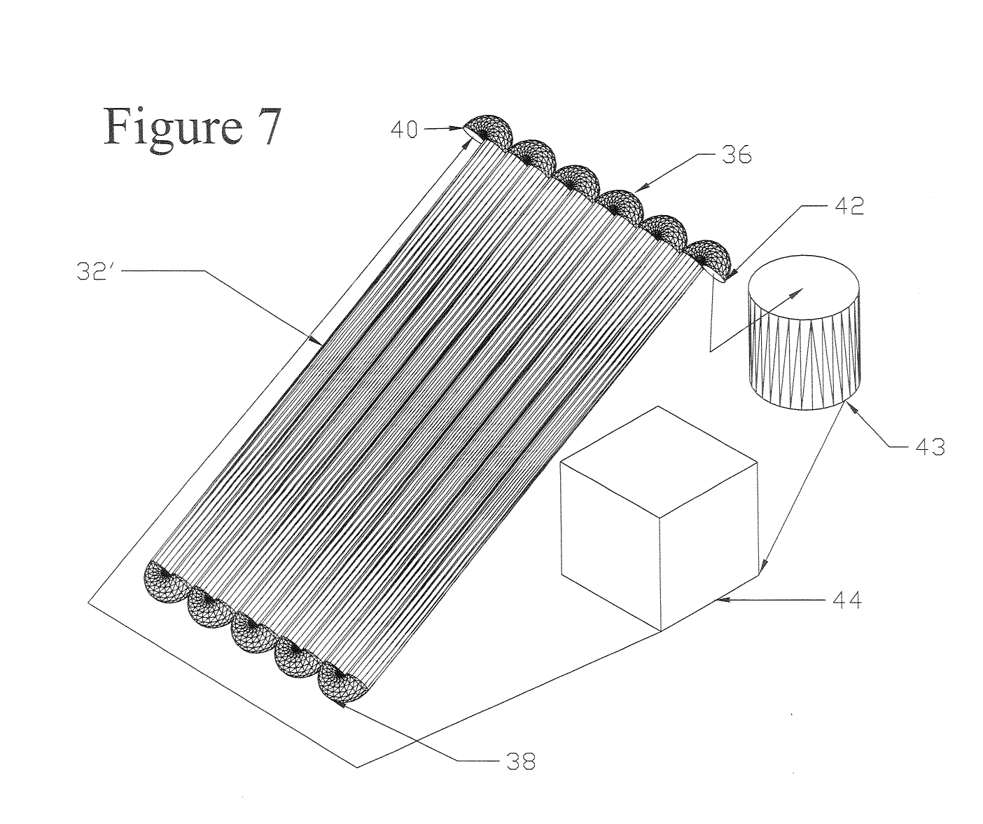

FIG. 6 is an example of how pipes 32 may be used over a rail road track 34. The pipes 32 filled with algae are strong and durable and may be connected together or left separate. FIG. 7 shows an example of a pipe system incorporating a flow-through design. In this representation, the pipes 32′ are connected together strategically on the top 36 and bottom 38 to encourage both to create containable sections in the case of a failure, and to create a flow with a beginning 40 and an end 42. The algae strainer/processor 44 is constantly recycling the cleaned water back into the system at the beginning 40. An optional reservoir 43 may be added to stabilize and monitor the water levels and to add to the water volume capacity of the system.

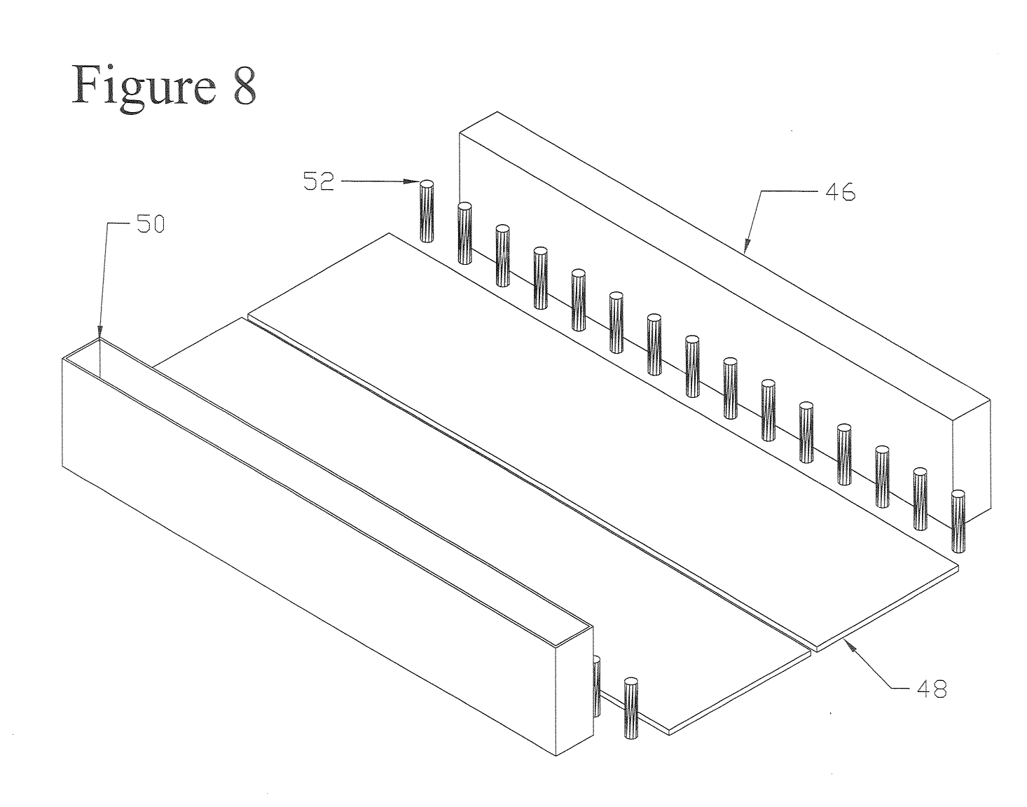

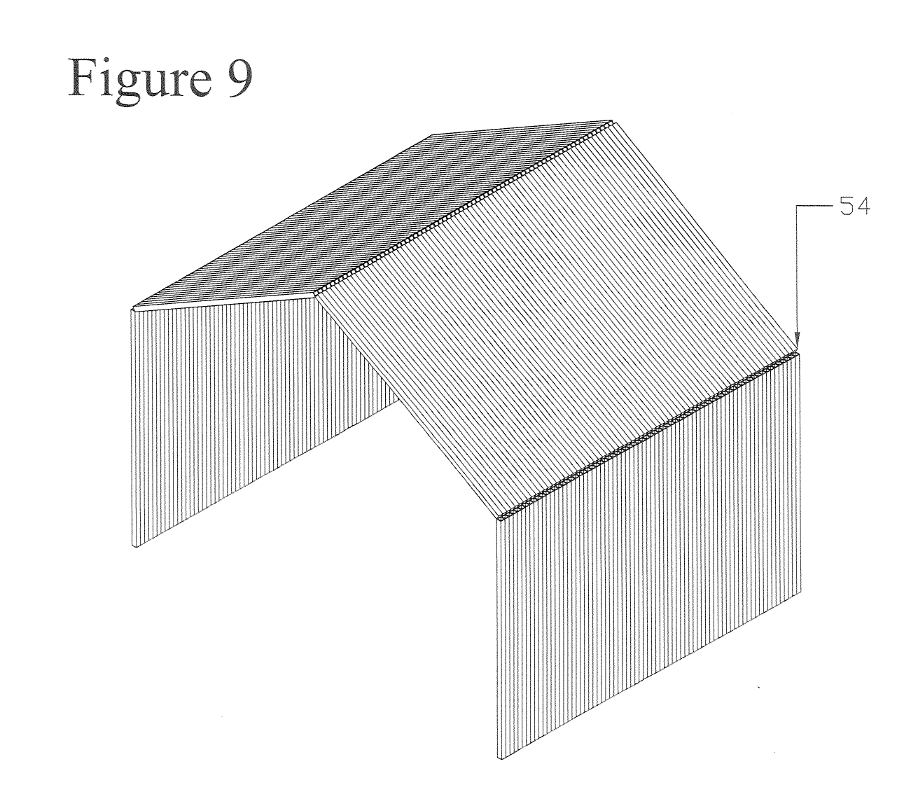

In FIG. 8, the tank 46 is acting as a wall and sound-buffer along an interstate highway 48. This tank may be left open on the top 50 for an open-loop system or it may be designed as a closed-loop system with a closed top 50. Road buffers or blockade posts 52 act to protect the tanks in the event of an accident. FIG. 9 exemplifies another embodiment, which utilizes thin tanks. In this figure, the tanks 54 are acting as wall and roof panels of a building. These panels are filled with growing algae communities.

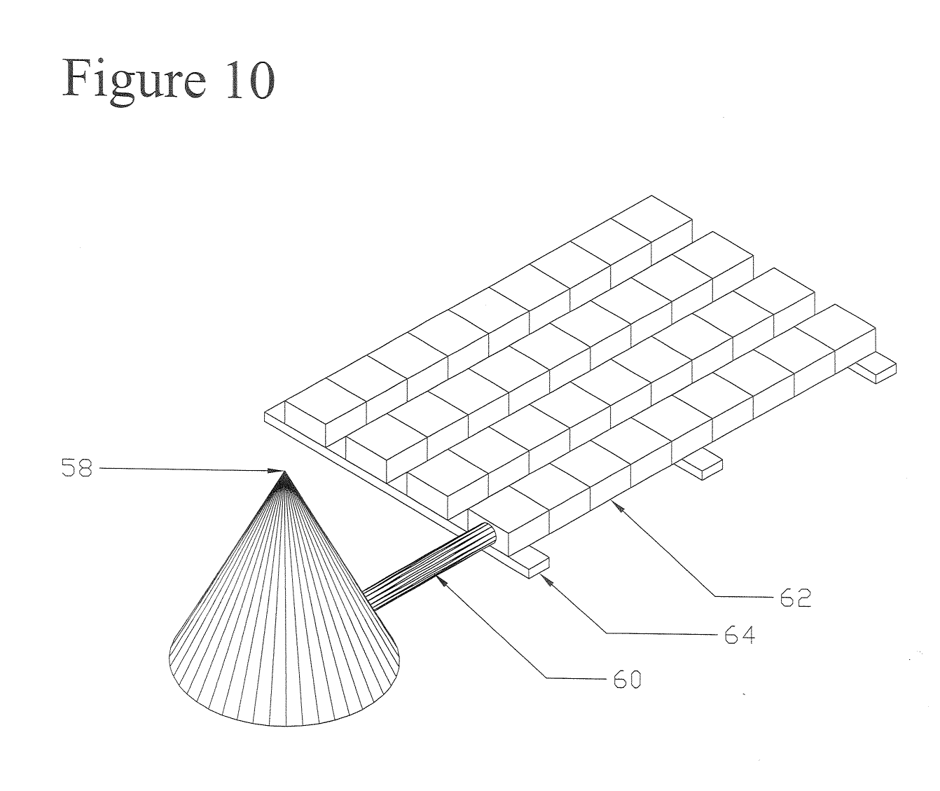

FIG. 10 shows a different application of the invention wherein the tank infrastructure is installed on top of a body of water. The tanks are anchored to the floor of the body of water or otherwise secured, and are designed to be submersible in areas that receive turbulent weather. In this embodiment, the tanks 62 are optionally installed with gaps between them to allow sunlight through and are carried by one or more pontoons or float members 64 floating on the ocean, a lake, or some other natural or manmade body of water. There is a pipe 60 connecting the tank to a local factory or refinery 58, or other source of carbon-dioxide rich gases that is used to bubble into the algae to increase algae production and reduce factory greenhouse emissions.

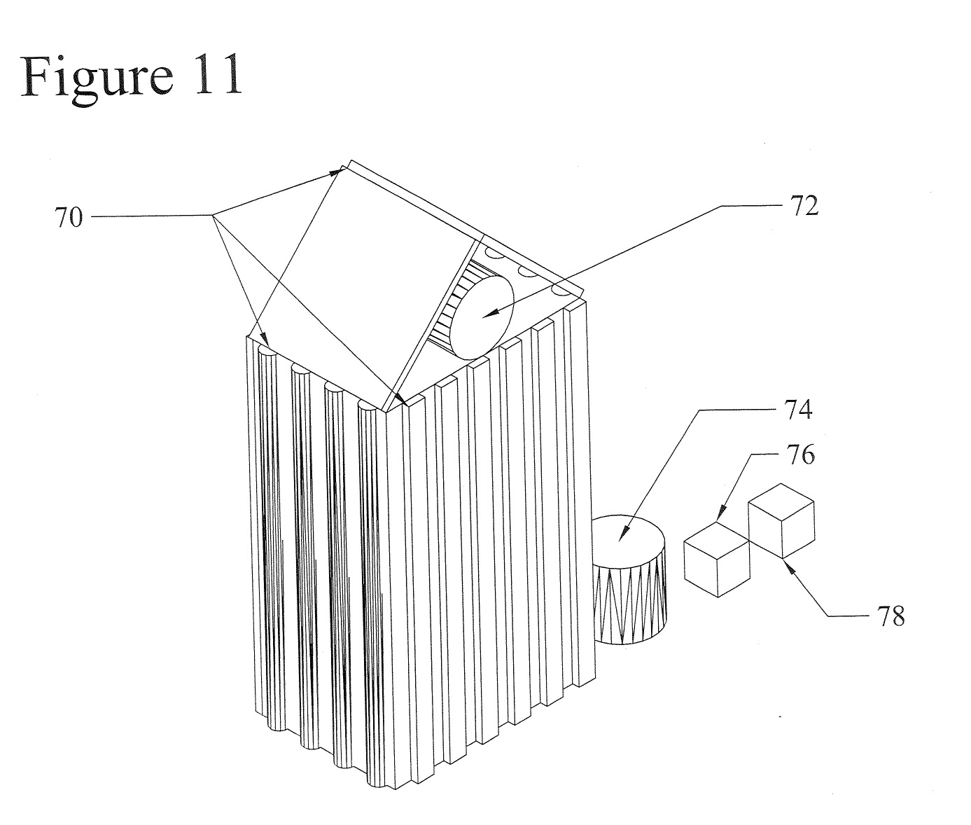

FIG. 11 displays an embodiment of the invention where tanks 70 filled with algae are mounted in, on or above a building in a closed system design. This system preferably is located near a natural abundance of salt water so that the salt water can be used to fill the tanks. An additional subsystem 72 is used to capture and condense fresh water from the top of the tanks that is evaporating from the salt water to be used potentially for drinking water or other personal use, and this system may also capture the high quality, oxygen-rich air from the tanks and pipe it into the building to improve the air quality people are breathing. A holding tank 74 may be added to the system nearby to hold extra water with algae. To process the algae, a processing station 76 is used to filter, dry and press the algae to produce oil for fuel. A diesel engine 78 may be used to produce electricity, heat and air conditioning for the building using the oil that is produced from the processing station 76. In this embodiment, if the building produces more oil than it uses in the diesel engine for heat, electricity, and air condition, then this building will act as a CO₂ sink and an oil and energy production facility. The diesel engine 76 may be replaced with a boiler or other device to produce a similar result.

While the invention has been described with reference to preferred and example embodiments, it will be understood by those skilled in the art that a variety of modifications, additions and deletions are within the scope of the invention, as defined by the following claims.

What is claimed is:

1. A method of absorbing solar energy and carbon dioxide, said method comprising:
mounting a container over a structure;
exposing the contents of said container to sunlight;
delivering air into said container; and
growing algae within said container.

2. The method of claim 1, wherein the structure is an existing element of infrastructure, selected from a roadway, a building, a railway, or a parking lot.

3. The method of claim 1, further comprising harvesting the algae grown in the container.

4. The method of claim 3, wherein the harvested algae is further processed to generate fuel.

5. The method of claim 1, further comprising collecting gases emitted from the growing algae.

6. A system for growing algae over human infrastructure, said system comprising:
a tank comprising a sunlight-transmissive top portion;
a quantity of algae-containing water disposed within said tank; and
means for delivering air into said tank; and
means for collecting algae from the water.

7. The system of claim 6, wherein at least the sunlight-transmissive top portion of the tank is ridged.

8. The system of claim 6, wherein at least a portion of the tank is oriented at an oblique angle to the horizontal.

9. The system of claim 6, further comprising means for controlling the temperature of the air delivered into the tank.

10. The system of claim 6, comprising a closed system.

11. The system of claim 6, comprising an open system.

12. The system of claim 6, wherein the tank comprises a series of clear piping segments.

13. The system of claim 6, wherein the tank comprises one or more roof or wall panels of a building.

14. The system of claim 6, wherein the tank comprises a conduit, and wherein the water circulates continuously through the conduit from a low algae concentration end to a high algae concentration end, and is collected at the high algae concentration.

15. The system of claim 6, wherein the tank comprises a sound buffer wall along a transportation corridor.

16. The system of claim 6, wherein the tank comprises a roof or wall panel of a building.

17. The system of claim 6, wherein the tank overlies manmade infrastructure.

18. The system of claim 6, wherein the tank is floated on a body of water.

19. A method of reducing greenhouse gases in the earth's atmosphere, said method comprising covering at least a portion of a component of human infrastructure with a vessel containing water, growing algae in the water contained in the vessel, and delivering atmospheric air into the vessel.

20. A method of reducing global warming, said method comprising covering at least a portion of a component of human infrastructure with a vessel containing water, exposing the vessel to sunlight, and growing algae in the water contained in the vessel.

21. A method of producing biofuel, said method comprising covering at least a portion of a component of human infrastructure with a vessel containing water, growing algae in the water contained in the vessel, and processing the algae into biofuel.

22. A building system comprising:
a container of algae-containing water, said container having at least one light-transmissive surface for generating algae growth within the water;
a water collection subsystem for collecting water vapor formed from evaporation of the water;
an air collection subsystem for collecting oxygen-rich air generated in the container;
a processing subsystem for producing fuel from the algae; and
a generator for producing power from the fuel.
